(19) United States
(12) Patent Application Publication
(10) Pub. No.: US 2026/0263211 A1
(43) Pub. Date: Sep. 10, 2026

(54) INTRAOCULAR LENS INJECTOR

(71) Applicant: ALCON INC., Fribourg (CH)

(72) Inventor: Kyle BROWN, Fort Worth, TX (US)

(21) Appl. No.: 19/555,181

(22) Filed: Mar. 3, 2026

Related U.S. Application Data
(60) Provisional application No. 63/767,878, filed on Mar. 6, 2025.

(51) Int. Cl. A61F 2/16

(52) U.S. Cl. CPC A61F 2/167; A61F 2/1678

(57) ABSTRACT

The present disclosure relates to an intraocular lens injector that includes an injector body and a nozzle coupled to the injector body to deliver an IOL to a surgical site. The injector body may include a compartment to house the IOL and receive a moveable plunger. A folding chamber may be coupled to the compartment. The folding chamber may be shaped to fold opposing portions of the IOL as the moveable plunger advances the IOL through the folding chamber to form an optic fold and to offset the optic fold from a vertical plane centered within and extending through the folding chamber.

### CROSS-REFERENCE TO RELATED APPLICATION

This application claims priority to U.S. Provisional Patent Application No. 63/767,878 filed on Mar. 6, 2026, the disclosure of which is incorporated herein by reference in its entirety.

### FIELD

The present disclosure relates to an intraocular lens injector.

### BACKGROUND

The human eye functions to provide vision by transmitting light through a clear outer portion called the cornea and focusing the image by way of a crystalline lens onto the retina at the back of the eye. The quality of the focused image depends on many factors including the size, shape, and length of the eye, and the shape and transparency of the cornea and the lens. When trauma, age or disease causes the lens to become less transparent, vision deteriorates because of the diminished light transmitted to the retina. This deficiency in the lens of the eye is medically known as a cataract. An accepted treatment for this condition is surgical removal of the lens and implantation of an artificial intraocular lens (IOL).

Cataractous lenses may be removed by a surgical technique called phacoemulsification. During this procedure, an incision is made in the anterior capsule that surrounds the lens such that the diseased lens may be removed. Once removed, an IOL injector may be used to deliver the IOL into the eye through the same incision used to remove the diseased lens.

### SUMMARY

According to certain embodiments, an intraocular lens (IOL) injector includes an injector body coupled to a nozzle that delivers an IOL to a surgical site. The injector body includes a compartment that houses an IOL and receives a moveable plunger. The injector body also includes a folding chamber coupled to the compartment. The folding chamber may be shaped to fold opposing portions of the IOL as the moveable plunger advances the IOL through the folding chamber to form an optic fold. The folding chamber may be shaped to offset the optic fold from a vertical plane centered within and extending through the folding chamber.

According to certain embodiments, a method may include positioning a nozzle of an intraocular lens (IOL) injector proximate a surgical site and folding an IOL such that opposing portions of the IOL form an optic fold that is offset from a vertical plane centered within and extending through the nozzle. The method further includes delivering the IOL to the surgical site.

According to certain embodiments, a method may include positioning a nozzle of an intraocular lens (IOL) injector proximate a surgical site and folding an IOL such that opposing portions of the IOL form an optic fold that is offset from a vertical plane centered within and extending through the nozzle. After folding the IOL, the method may further include advancing a plunger to deliver the IOL into the surgical site by ejecting the IOL out of the nozzle.

### BRIEF DESCRIPTION OF THE DRAWINGS

Example embodiments will be described and explained with additional specificity and detail through the accompanying drawings in which:

FIG. 1A illustrates an example intraocular lens injector including a handpiece body and a movable plunger;

FIG. 1B illustrates an exploded view of the example intraocular lens injector of FIG. 1A;

FIG. 2 illustrates an example intraocular lens;

FIG. 3A illustrates inserting an example intraocular lens into a compartment of an example intraocular lens injector;

FIGS. 3B–3D illustrates the example intraocular lens injector of FIG. 3A in use at varying stages of IOL advancement and folding;

FIG. 4A illustrates a top, cross-sectional view of an example intraocular lens injector;

FIG. 4B illustrates a top, cross-sectional view of the example intraocular lens injector of FIG. 4A with a modified first sidewall;

FIG. 4C illustrates a cross-sectional view of the IOL injector 400 taken along the 4C–4C cutting plane of FIG. 4A.

FIG. 4D illustrates a cross-sectional view of the IOL injector 400 taken along the 4D–4D cutting plane of FIG. 4A; and

FIG. 5 is a flow diagram illustrating a method for delivering an IOL to a surgical site.

All according to one or more embodiments of the present disclosure.

### DETAILED DESCRIPTION

The present disclosure relates to apparatuses and methods for delivering an intraocular lens (IOL) to a surgical site within a human eye using an intraocular lens injector. The IOL may be folded within an IOL injector before delivery so that the lens may be delivered through an incision in the cornea. In some embodiments, the IOL may include an optic and leading and trailing haptics connected to opposing portions of the optic. The IOL injector may fold opposing portions of the optic to form a fold. Traditionally, IOL injectors have been configured to position the fold such that the fold faces upwards and aligns with the top center of the IOL injector (e.g., at the 12 o’clock position). Positioning the fold at the 12 o’clock position provides the surgeon with a direct line of visibility of the fold when looking downwards at the IOL injector. This may allow the surgeon to determine if the folding configuration of the IOL may benefit from rotation of the IOL injector during delivery of the IOL to mitigate tilting of the IOL within the eye or if the IOL may be delivered without rotating the IOL injector. The determination of whether a rotation is beneficial may be based on the position of the leading haptic, the position of the trailing haptic, and/or the position of the fold. For example, the leading haptic may be tucked within the fold of the optic, and, in this configuration, the surgeon may determine that the IOL may be delivered without rotating the IOL injector.

In some instances, the IOL may become tilted in the eye upon delivery. A delivered IOL is considered to be tilted if the IOL is not flat, planar, and/or parallel relative to the iris. Lens tilt may occur due to improper rotation of the IOL injector or improper timing of the rotation during delivery. Lens tilt may also occur in IOL folding configurations that do not utilize IOL rotation during delivery because a portion of the optic connected to the leading haptic may unfold faster than the opposing portion of the optic connected to the trailing haptic. This phenomenon combined with IOL injectors that are configured to align the optic fold with the top center of the IOL injector (e.g., at the 12 o’clock position) may result in the IOL being tilted upon delivery in the direction of the portion of the optic connected to the leading haptic. Thus, where one portion of the optic unfolds faster than an opposing portion of the optic, traditional methods of positioning the fold to face upward at the top center of the IOL injector may be disadvantageous because this position may lead to an increased frequency of tilted lenses.

Tilted IOLs may present an issue during cataract surgery and IOL implantation because the surgeon may undertake unusual, aggressive, and difficult manipulations of the IOL post-delivery to position the IOL in proper alignment. In some of these instances, the surgeon may lose orientation and move the optic in the wrong direction, resulting in a lens that is upside down within the capsular bag of the eye. Furthermore, a tilted IOL may not focus light properly onto the retina, which may result in blurry or distorted vision.

One or more embodiments of the present disclosure relate to an IOL injector that mitigates IOL tilt, for example, by offsetting the folded orientation of the optic from the traditional 12 o’clock position prior to delivery into an eye. An example IOL injector includes an injector body and a nozzle coupled to the injector body. In particular, in some embodiments, the injector body includes a compartment to house an IOL and receive a moveable plunger and a folding chamber coupled to the compartment. The folding chamber is shaped to fold opposing portions of an optic of the IOL as the moveable plunger advances the IOL through the folding chamber to form an optic fold and offset the optic fold from a vertical plane centered within and extending through the folding chamber. Thus, the IOL injector may compensate for one portion of the optic unfolding faster during delivery within the eye than another portion of the optic by offsetting the optic fold of the IOL from the vertical plane. Such compensation may allow the optic fold to be delivered planar or flat to the direction of incoming light and may reduce the frequency of or remove altogether surgeons rotating the injector during ejection of the lens into the eye. Therefore, one or more embodiments of the present disclosure may improve ophthalmic surgical devices by mitigating IOL tilt allowing for safer and more effective IOL implantation.

Embodiments of the present disclosure may be utilized with any suitable system, apparatus or device that includes an IOL injector. Example IOL delivery devices with which some embodiments may be implemented include the MONARCH® III delivery systems available from ALCON® or the MONARCH® IV handpiece available from ALCON®. Some embodiments may be implemented with other IOL delivery devices from ALCON® and/or from other vendors/suppliers. Additionally or alternatively, some embodiments may include preloaded cartridges or injectors, such as the ULTRASERT® or AUTONOME® from ALCON®.

The embodiments of the present disclosure will be explained with reference to the accompanying figures. It is to be understood that the figures are diagrammatic and schematic representations of such example embodiments, and are not limiting, nor are they necessarily drawn to scale. In the figures, features with like numbers indicate like structure and function unless described otherwise. Further, one or more of the figures and accompanying descriptions are given with respect to ophthalmic surgical systems. However, such uses are not meant to be limiting such that the devices described may be used in any number of different contexts and applications where they may be helpful or applicable.

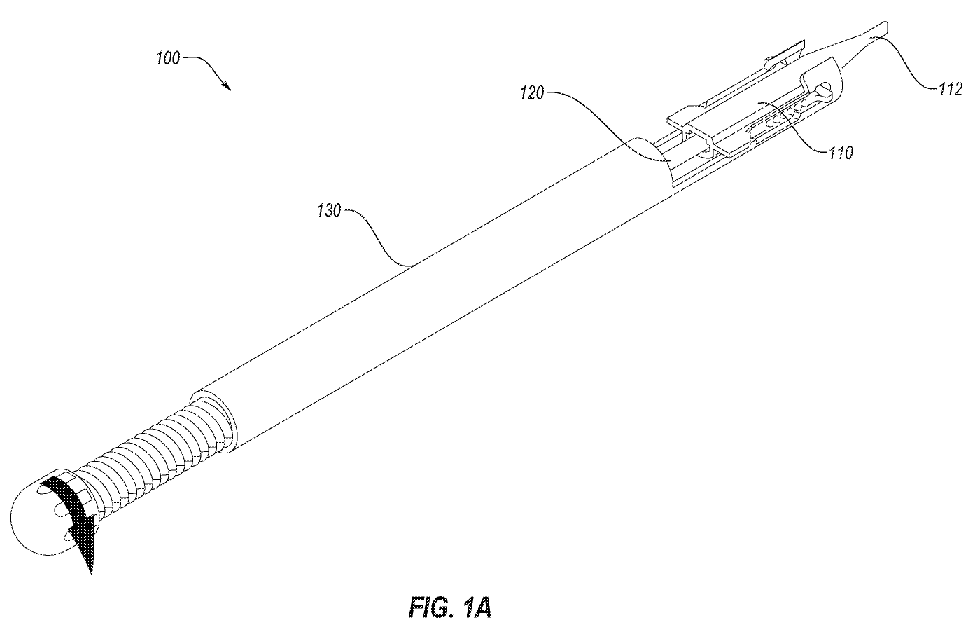

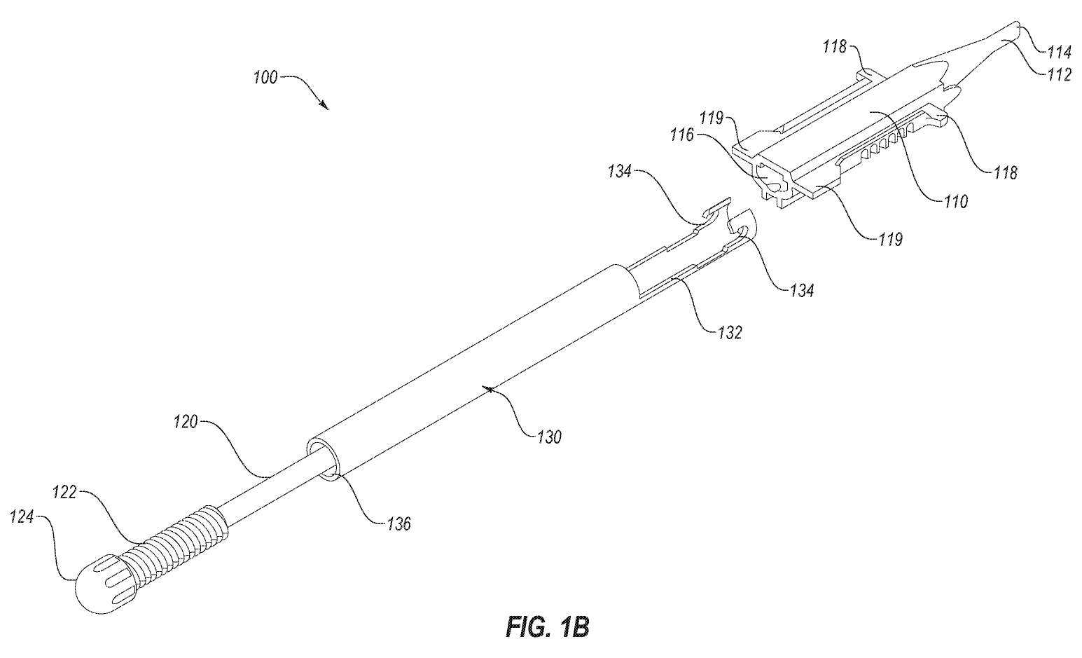

FIG. 1A and FIG. 1B respectively illustrate a perspective view of an intraocular lens (IOL) injector 100 and an exploded view of the IOL injector 100.

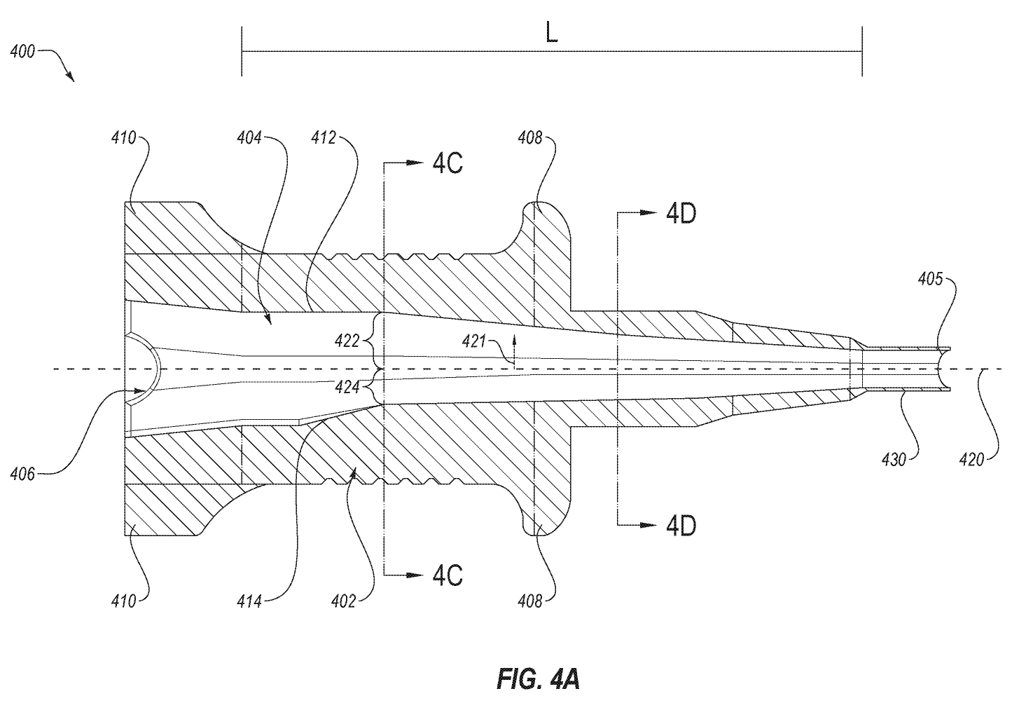

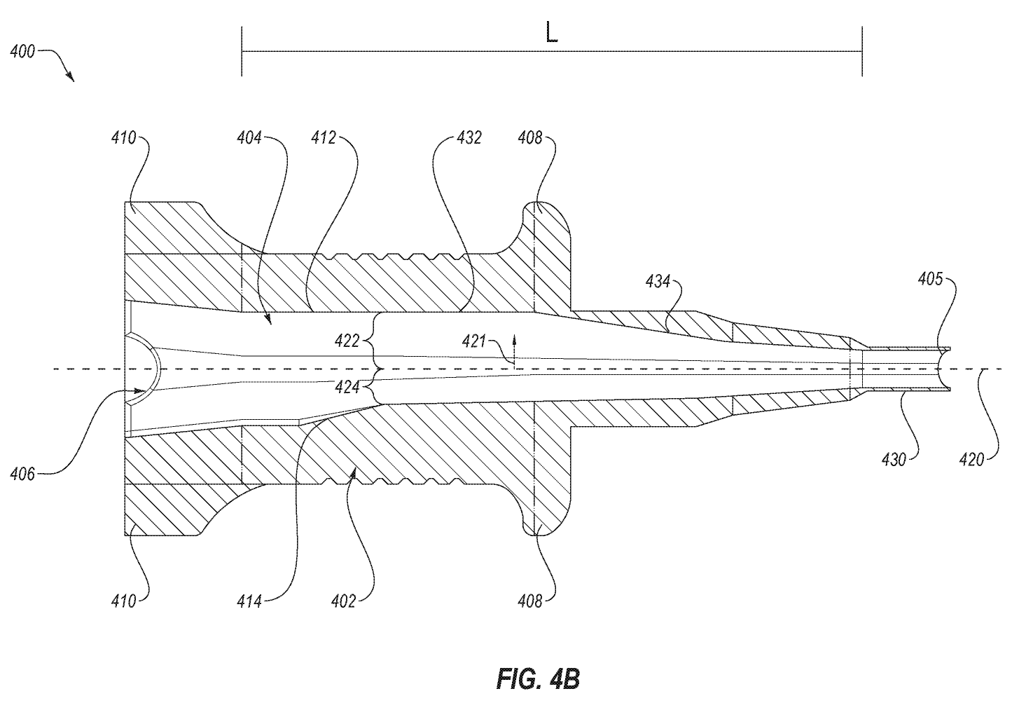

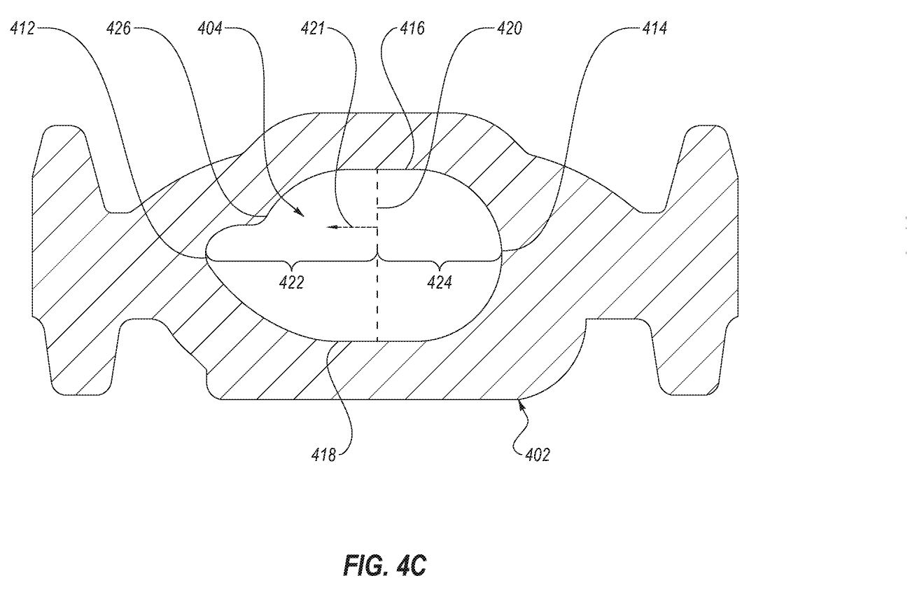

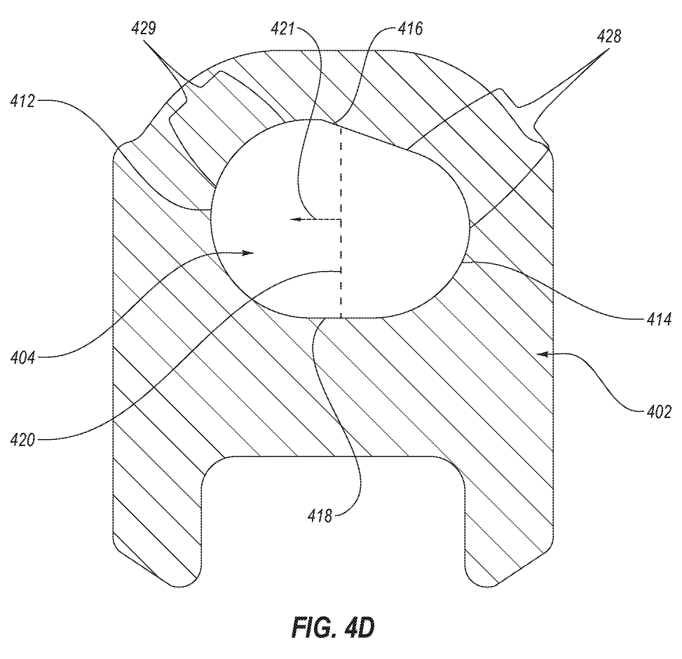

The IOL injector 100 includes an injector body 110 and a nozzle 112 coupled to the injector body 110. The nozzle 112 may be configured to deliver an intraocular lens (IOL) to, for example, a surgical site such as an eye. The injector body 110 includes a compartment 116 to house an IOL and receive a moveable plunger 120. The injector body 110 also includes a folding chamber (not shown) coupled to the compartment 116. The folding chamber is shaped to fold opposing portions of an optic of the IOL as the moveable plunger 120 advances the IOL through the folding chamber to form an optic fold and to offset the optic fold from a vertical plane centered within and extending through the folding chamber and/or the nozzle 112. In some embodiments, such as illustrated in FIG. 4A, the compartment 116, the folding chamber, and the nozzle 112 may be a continuous channel within the injector body 110.

In some embodiments, the IOL injector 100 may further include a handpiece body 130 coupled to the injector body 110. In some embodiments, the handpiece body 130 may include a receptacle 132 and slots 134. In some embodiments, the injector body 110 may further include a first set of tabs 118 and a second set of tabs 119. In some of these embodiments, each tab of the first set of tabs 118 may extend from opposing sides of the injector body 110 and may be received within the slots 134. In some of these embodiments, each tab of the second set of tabs 119 may extend from opposing sides of the injector body 110 and may rest on opposing sides of the receptacle 132 when the first set of tabs 118 are inserted within slots 134 to stabilize the injector body 110 while the IOL is delivered into, for example, a human eye.

In some embodiments and as shown in FIGS. 1A and 1B, the injector body 110 may be a cartridge that may be coupled to the handpiece body 130. In some embodiments, the injector body 110 may be detachable from the handpiece body 130. For example, the first set of tabs 118 may be removed from the slots 134 and the injector body 110 may be detached from the handpiece body 130. In some embodiments, the injector body 110 may be integral with the handpiece body 130 such that the injector body 110 and the handpiece body 130 are one unit.

In some embodiments, the IOL injector 100 may include a moveable plunger 120 disposed at least partially within the handpiece body 130 and axially translatable relative to the handpiece body 130. In some of these embodiments, the moveable plunger 120 may be axially translatable relative to the handpiece body 130 via an adjustment knob 124. The adjustment knob 124 may engage the moveable plunger 120 to translate the moveable plunger 120 axially forward (i.e. in a direction toward and/or into the injector body 110) or axially backward (i.e., in a direction away from and/or out of the injector body 110) relative to the handpiece body 130.

A proximal end 136 of the handpiece body 130 may include internal threads (not shown), which may engage external threads 122 on a shaft of the adjustment knob 124. Due to the engagement between the internal threads and the external threads 122, rotating the adjustment knob 124 relative to the handpiece body 130 in one direction (e.g., clockwise in this example) may cause the adjustment knob 124 to advance or translate axially into or toward the handpiece body 130 as the external threads 122 are threaded into the internal threads. On the other hand, rotating the adjustment knob 124 relative to the handpiece body 130 in the other direction (e.g., counterclockwise in this example) may cause the adjustment knob 124 to retreat or translate axially away from the handpiece body 130 as the external threads 122 are threaded out of the internal threads.

In some embodiments, the external threads 122 and the internal threads may be threaded in a reverse orientation compared to the preceding example (e.g., left-hand threaded). In these embodiments, rotating the adjustment knob 124 counterclockwise relative to the handpiece body 130 may cause the adjustment knob 124 to advance or translate axially into or toward the handpiece body 130 and rotating the adjustment knob 124 clockwise may cause the adjustment knob 124 to retreat or translate axially away from the handpiece body 130.

In operation, an IOL may be inserted into the injector body 110 at a proximal end of the compartment 116. The first set of tabs 118 may be placed within the slots 134 and the second set of tabs 119 may rest on the receptacle 132. A surgeon may then position the nozzle 112 proximate the surgical site. The adjustment knob 124 may be rotated in one direction (e.g., clockwise) causing the adjustment knob 124 to advance toward the handpiece body 130, to activate the moveable plunger 120, and to translate the moveable plunger 120 axially forward in a direction from the proximal end 136 of the handpiece body 130 toward the injector body 110. The adjustment knob 124 may continue to be rotated in the same direction to translate the moveable plunger 120 into the compartment 116 where the moveable plunger 120 engages the IOL. The IOL may be advanced by the moveable plunger 120 through the folding chamber by further rotating the adjustment knob 124 in the same direction. The folding chamber may fold opposing portions of the optic of the IOL to form an optic fold and offset the optic fold from a vertical plane (not shown) centered within and extending through the folding chamber and/or the nozzle 112. The IOL may be advanced by the moveable plunger 120 into the nozzle 112 by rotating the adjustment knob 124 further in the same direction. When the IOL enters the nozzle 112, the surgeon may stop to visually confirm that the optic fold is offset from the vertical plane. The adjustment knob 124 may be further rotated in the same direction to deliver the IOL through the opening 114 of the nozzle 112 to, for example, a human eye at the surgical site.

Modifications, additions, or omissions may be made to the IOL injector 100 of FIGS. 1A and 1B without departing from the scope of the present disclosure. For example, while the handpiece body 130 is described as including an adjustment knob 124, other mechanisms may be utilized to axially translate the moveable plunger 120 such that the IOL is advanced from the compartment 116, through the folding chamber, through the nozzle 112, and into, for example, a human eye at the surgical site. For example, the moveable plunger 120 may include flanges or tabs formed at the proximal end 136 of the handpiece body 130, which may be manipulated by the fingers, thumb, or hand of a surgeon to advance the moveable plunger 120 through the handpiece body 130 and through the injector body 110 to deliver the IOL to the surgical site (e.g., in a similar fashion as a syringe). In another example, an electric motor may be coupled to the IOL injector 100, which may axially translate the moveable plunger 120 through the handpiece body 130 and through the injector body 110 to deliver the IOL to the surgical site.

Furthermore, the first set of tabs 118, slots 134, the second set of tabs 119, and the receptacle 132 demonstrate one example of how the injector body 110 may be coupled to the handpiece body 130, but any other method of coupling the injector body 110 to the handpiece body 130 may be implemented. For example, the injector body 110 and the handpiece body 130 may both be threaded, and the injector body 110 and the handpiece body 130 may threadably engage with one another to couple the injector body 110 to the handpiece body 130. In another example, the injector body 110 and the handpiece body 130 may be connected to each other via a male-female configuration such as a tab-and-recess, pin-and-box, tongue-and-groove, key-and-keyseat, or the like. As discussed previously, the injector body 110 and the handpiece body 130 may be integral and may not require coupling with one another.

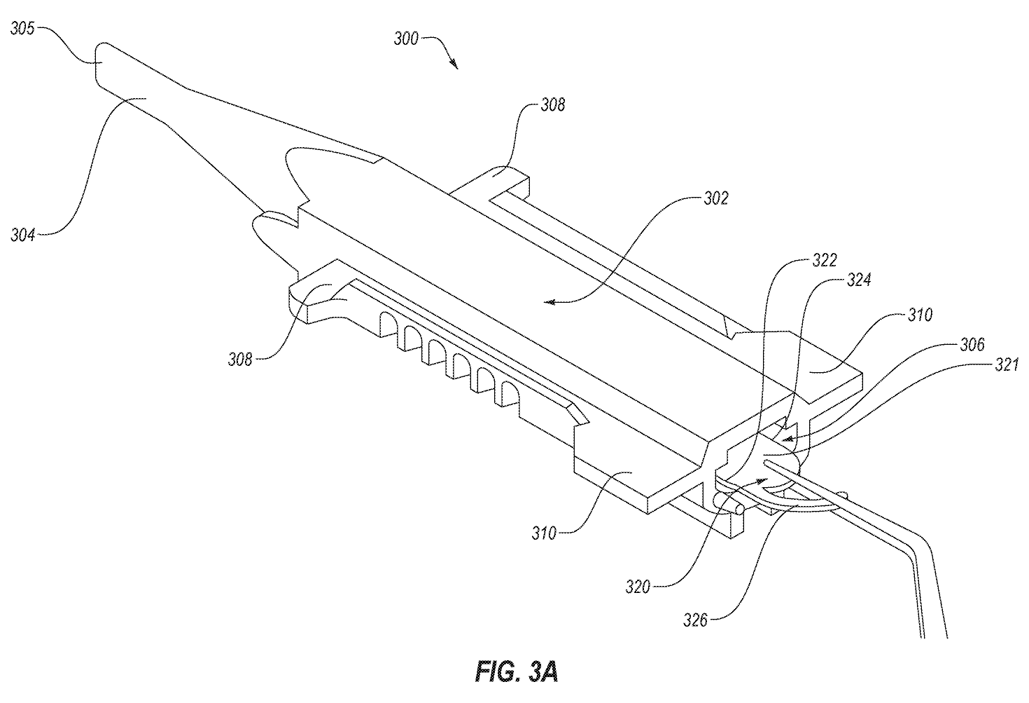

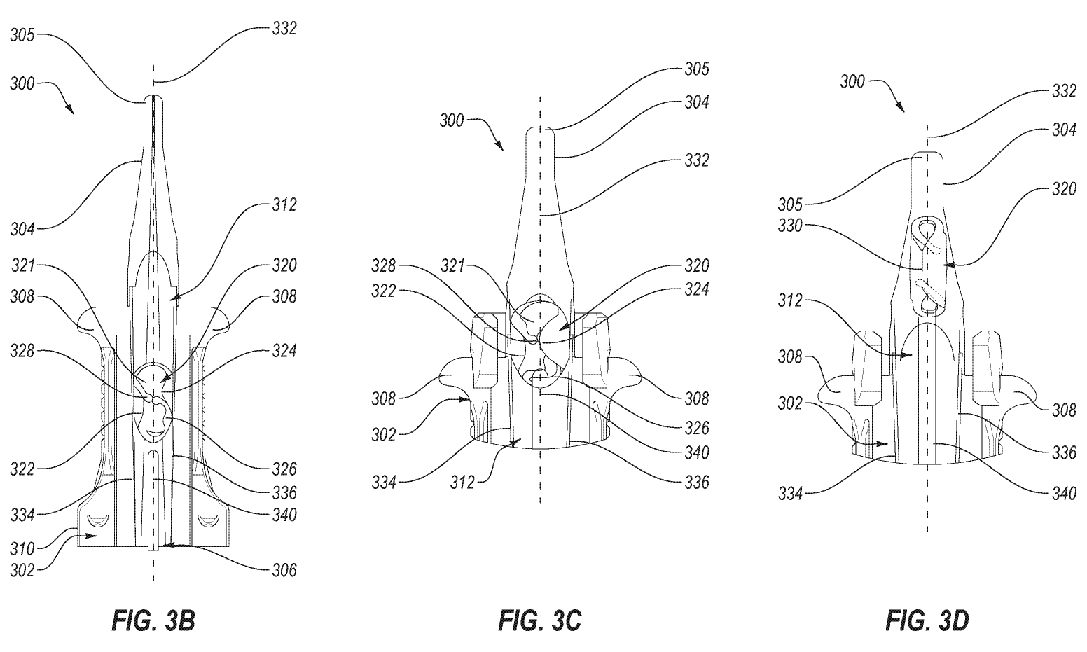

In addition, the components of the IOL injector 100 may be similar to and perform similar functions as similarly named components or elements described throughout the description and vice versa. The folding chamber of the IOL injector 100 may also include any or all of the features described with reference to FIGS. 3A–3D and FIGS. 4A–4D.

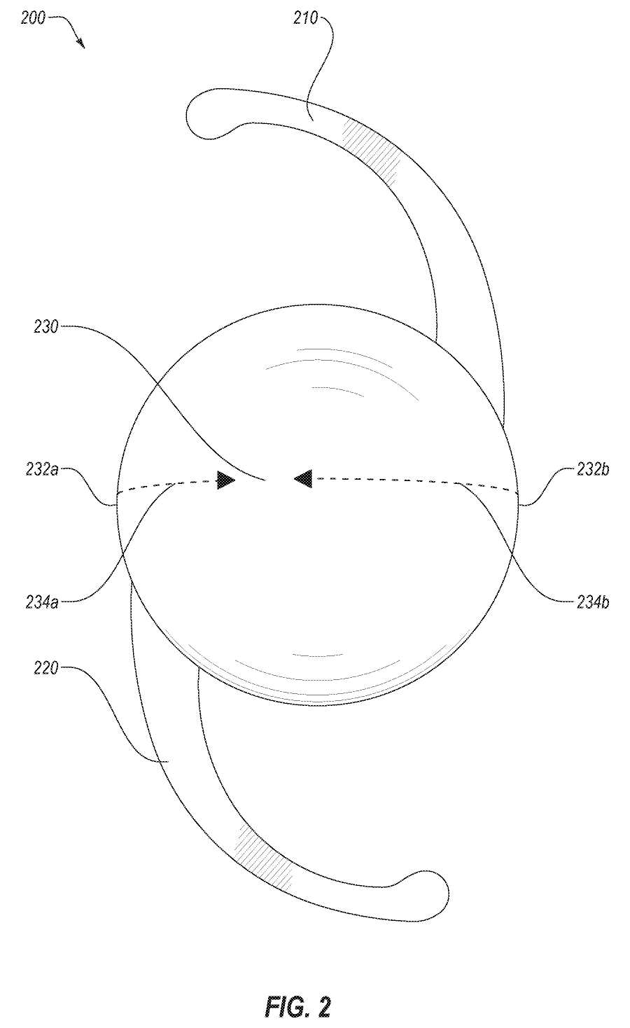

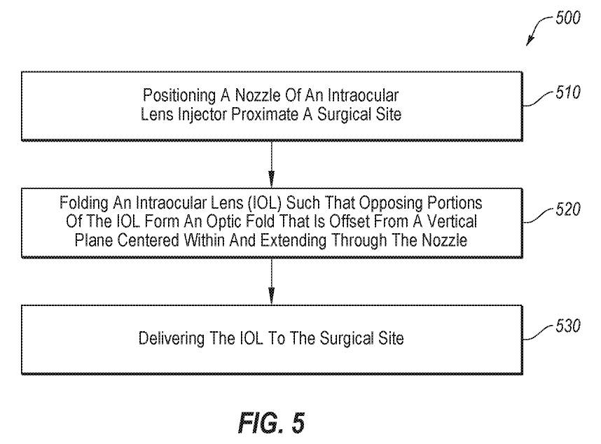

FIG. 2 illustrates an example intraocular lens (IOL) 200 that may be folded in the folding chamber of the example intraocular lens injectors of FIGS. 1A–1B, 3A–3D, 4A–4D and/or in the method of FIG. 5. The IOL 200 may include a leading haptic 210, a trailing haptic 220, and an optic 230. The optic 230 may include a first opposing portion 232a on the side of (e.g., proximate) where the trailing haptic 220 is coupled to the optic 230, and a second opposing portion 232b on the side of (e.g., proximate) where the leading haptic 210 is coupled to the optic 230. The first opposing portion 232a and the second opposing portion 232b may be folded inwards by the folding chamber (shown by arrows 234a and 243b, respectively).

In some embodiments, the optic 230, the leading haptic 210, and the trailing haptic 220 may be integrally formed out of a single piece of material. In some embodiments, the leading haptic 210 and the trailing haptic 220 may be formed separately from the optic 230, the optic 230 may be formed out of another material (or separately of the same material as the haptics 210, 220), and the optic 230, the leading haptic 210, and the trailing haptic 220 may be coupled together prior to delivery into a surgical site. In some embodiments, the optic 230, the leading haptic 210, and the trailing haptic 220 may be fixedly secured to each other prior to insertion into an IOL injector.

In operation, the IOL 200 may be inserted into a compartment of an injector body of an IOL injector. In some embodiments, the IOL 200 may be inserted by the end-user of the IOL injector (e.g., a surgeon, doctor, nurse, physician assistant, nurse practitioner, or other healthcare professionals). In some embodiments, the IOL 200 may be inserted into the compartment prior to the end-user receiving the IOL injector. For example, the IOL may be inserted into the compartment 116 of FIG. 1 during manufacturing or otherwise prior to distribution to the end-user.

An opening in a nozzle of the IOL injector such as the opening 114 of the nozzle 112 of FIG. 1 may be positioned proximate a surgical site. The leading haptic 210 may be in a leading or delivery side position such that the leading haptic 210 is the first portion of the IOL 200 to enter the surgical site, and the trailing haptic 220 may be in a trailing position such that the trailing haptic 220 is the last portion of the IOL 200 to enter the surgical site. A moveable plunger may be advanced into the compartment and engage the IOL 200 at the trailing haptic 220 and/or the optic 230. The moveable plunger may advance the IOL 200 through the folding chamber such that the opposing portions 232a and 232b of the optic 230 are folded inwards (as shown by arrows 234a and 234b, respectively) to form an optic fold (not shown). In some embodiments, the leading haptic 210 and the trailing haptic 220 may be tucked within the optic fold such that a distal end of the haptics are positioned within the optic fold as the IOL 200 advances through the folding chamber. In some embodiments, one or both of the leading haptic 210 or the trailing haptic 220 may not be tucked within the optic fold. The folding chamber may offset the optic fold from a vertical plane centered within and extending through the folding chamber and/or the nozzle. The offset of the optic fold is discussed in more detail with reference to FIGS. 3A-3D and with reference to FIGS. 4A-4D.

As the IOL 200 is delivered through the opening of the nozzle to, for example, a human eye at a surgical site, the leading haptic 210 may guide the IOL 200 through an incision in the eye at the surgical site and contact one side of the capsular bag in the eye. The trailing haptic 220 may enter the eye after the leading haptic 210 and contact an opposite side of the capsular bag as the leading haptic 210 to maintain the orientation of the optic 230 within the capsular bag in the eye. During delivery, the second opposing portion 232b of the optic 230 may unfold faster than the first opposing portion 232a of the optic 230 because the second opposing portion 232b is coupled to the leading haptic 210, which enters the surgical site first. The first opposing portion 232a is coupled to the trailing haptic 220, which enters the surgical site last. The first opposing portion 232a may unfold slower than the second opposing portion 232b because the trailing haptic 220 is contained within the IOL injector for a short period while the leading haptic 210 has already exited the IOL injector. Thus, if the optic fold is positioned vertically with no offset (e.g., the 12 o’clock position), the IOL 200 may be tilted upon delivery in the direction of the second opposing portion 232b. Because the folding chamber offsets the optic fold from a vertical plane, for example in the direction of the first opposing portion 232a, the risk of the IOL 200 being tilted upon delivery may be mitigated because offsetting the optic fold may compensate for the second opposing portion 232b unfolding faster than the first opposing portion 232a.

FIG. 3A illustrates inserting an intraocular lens (IOL) 320 into a compartment 306 of an IOL injector 300. The IOL injector 300 may include an injector body 302 and a nozzle 304 coupled to the injector body 302. The injector body 302 of the IOL injector 300 includes a compartment 306. The compartment 306 houses an IOL 320 and receives a moveable plunger (not shown in FIG. 3A).

In operation, the IOL 320 may be inserted into the compartment 306 as shown in FIG. 3A. In some embodiments, the IOL 320 may be inserted into the compartment 306 at a manufacturing facility or otherwise before an end-user receives the IOL injector 300. In some embodiments, the IOL 320 may be inserted by the end-user, for example a surgeon, doctor, nurse, physician assistant, nurse practitioner, or other healthcare professionals. For example, as shown in FIG. 3A, the IOL 320 may be inserted using forceps before surgery. The IOL 320 may include an optic 321 having opposing portions 322 and 324, a trailing haptic 326, and a leading haptic (not shown in FIG. 3A). The leading haptic may extend from the second opposing portion 324 and the trailing haptic 326 may extend from the first opposing portion 322. In some embodiments, the leading haptic (not shown) may extend from the first opposing portion 322 and the trailing haptic 326 may extend from the second opposing portion 324.

As shown in FIG. 3A, the IOL injector 300 is a cartridge that may include a first set of tabs 308 and a second set of tabs 310. In some of these embodiments, each tab of the first set of tabs 308 may extend from opposing sides of the injector body 302. In some embodiments, each tab of the second set of tabs 310 may extend from opposing sides of the injector body 302. In some embodiments, the injector body 302 may be coupled and uncoupled with a handpiece body via the first set of tabs 308 and the second set of tabs 310. For example, the first set of tabs 308 may engage with slots 134 in the handpiece body 130 of FIG. 1 and the second set of tabs 310 may rest on a receptacle 132 of the handpiece body 130 of FIG. 1 to stabilize the IOL injector 300. In some embodiments, the first set of tabs 308 and/or the second set of tabs 310 may be omitted.

FIGS. 3B–3D illustrate the IOL injector 300 in use at varying stages of IOL advancement. FIG. 3B illustrates the IOL injector 300 at a beginning stage of IOL advancement. FIG. 3C illustrates the IOL injector 300 at a middle stage of IOL advancement. FIG. 3D illustrates the IOL injector 300 at an end-stage of IOL advancement where an optic fold 330 has been formed, at least in part, by a folding chamber 312.

As shown in FIGS. 3B–3D, the injector body 302 of the IOL injector 300 includes the folding chamber 312. The folding chamber 312 may be shaped to fold the first opposing portion 322 and the second opposing portion 324 of the optic 321 of the IOL 320 as the moveable plunger 340 advances through the folding chamber 312 to form an optic fold 330. The folding chamber 312 may be shaped to offset the optic fold 330 from a vertical plane 332 centered within and extending through the folding chamber 312 and/or the nozzle 304.

In some embodiments, the folding chamber 312 may be defined, at least in part, by a first sidewall 334, a second sidewall 336 opposite the first sidewall 334, a third sidewall (not shown) extending between the first sidewall 334 and the second sidewall 336, and a fourth sidewall (not shown) opposite the third sidewall and extending between the first sidewall 334 and the second sidewall 336. In some embodiments, the third sidewall may be an upper sidewall or otherwise form an upper portion of the folding chamber 312. In some embodiments, the fourth sidewall may be a lower sidewall or otherwise form a lower portion of the folding chamber 312. In some embodiments, the folding chamber 312 may have a substantially rounded cross-section such that each of the interfaces between the first sidewall 334, the second sidewall 336, the third sidewall, and the fourth sidewall are rounded.

In operation, the moveable plunger 340 may be activated to advance the IOL 320 into the folding chamber 312. As shown in FIG. 3B and FIG. 3C, the moveable plunger 340 advances the IOL 320 through the folding chamber 312 and the first sidewall 334 may fold the first opposing portion 322 of the IOL 320 inwards toward the vertical plane 332 (e.g., in the direction illustrated by the arrow 234a shown in FIG. 2) and the second sidewall 336 may fold the second opposing portion 324 of the IOL 320 inwards toward the vertical plane 332 (e.g., in the direction illustrate by the arrow 234b shown in FIG. 2).

As shown in FIG. 3D, the moveable plunger 340 advances the IOL 320 through the folding chamber 312 into the nozzle 304, and the IOL 320 may be folded until the opposing portions 322 and 324 form the optic fold 330, which is offset from the vertical plane 332. In some embodiments, the optic fold 330 may be offset from the vertical plane 332 toward the first sidewall 334. In some embodiments, the optic fold 330 may be offset from the vertical plane 332 toward the second sidewall 336. In some embodiments, the optic fold 330 may be offset from the vertical plane 332 towards a side on which the trailing haptic 326 couples to the optic 321. In some embodiments, the folding chamber 312 may be shaped to tuck the leading haptic 328 within the optic fold 330 and/or to tuck the trailing haptic 326 within the optic fold 330.

An opening 305 of the nozzle 304 of the IOL injector 300 may be positioned proximate a surgical site before or after the optic fold 330 has been formed. The leading haptic 328 may be in a leading or delivery side position such that the leading haptic 328 is the first portion of the IOL 320 to enter the surgical site, and the trailing haptic 326 may be in a trailing position such that the trailing haptic 326 is the last portion of the IOL 320 to enter the surgical site. The moveable plunger 340 may be further advanced (beyond that shown in FIG. 3D) to deliver the IOL 320 through the opening 305 of the nozzle 304 to, for example, a human eye at a surgical site.

As shown in FIGS. 3B–3D, the leading haptic 328 extends from the second opposing portion 324 and the trailing haptic 326 extends from the first opposing portion 322. In these and other embodiments, during delivery of the IOL 320, the second opposing portion 324 of the optic 321 may unfold faster than the first opposing portion 322 of the optic 321 because the second opposing portion 324 is coupled to the leading haptic 328, which enters the surgical site first. The first opposing portion 322 is coupled to the trailing haptic 326, which enters the surgical site last. The first opposing portion 322 may unfold slower than the second opposing portion 324 because the trailing haptic 326 is contained within the IOL injector 300 for a short period while the leading haptic 328 has already exited the IOL injector 300 through the opening 305. Thus, if the optic fold 330 is positioned vertically (e.g., at the 12 o’clock position) so as to be aligned with the vertical plane 332, the IOL 320 may be tilted upon delivery in the direction of the second opposing portion 324. To compensate for the delayed unfolding of the first opposing portion 322 compared to the second opposing portion 324, the folding chamber 312 may be shaped to offset the optic fold 330 from the vertical plane 332 toward the first sidewall 334, which may mitigate tilting of the IOL 320 upon delivery.

In some embodiments, the folding chamber 312 may be shaped to offset the optic fold 330 about 15 degrees, about 20 degrees, about 30 degrees, about 40 degrees, about 45 degrees, about 50 degrees, about 60 degrees, about 75 degrees, or other values, from the vertical plane 332 toward the first sidewall 334. In some embodiments, the folding chamber 312 may be shaped to offset the optic fold 330 between about 15 degrees and about 75 degrees from the vertical plane 332 toward the first sidewall 334. In some embodiments, the folding chamber 312 may be shaped to offset the optic fold 330 between about 30 degrees and about 60 degrees from the vertical plane 332 toward the first sidewall 334. In some embodiments, the folding chamber 312 may be shaped to offset the optic fold 330 less than 15 degrees from the vertical plane 332 toward the first sidewall 334. In some embodiments, the folding chamber 312 may be shaped to offset the optic fold 330 from the vertical plane 332 toward the first sidewall 334 in any range using any of the preceding values as endpoints, including ranges that include the value as an endpoint and the vertical plane 332 as an endpoint (e.g., less than 15 degrees, less than 30 degrees, less than 60 degrees, between 15 degrees and 45 degrees, between 20 degrees and 50 degrees, etc.).

In some embodiments, inverse to the IOL 320 illustrated in FIGS. 3A-3D, the leading haptic 328 may extend from the first opposing portion 322 and the trailing haptic 326 may extend from the second opposing portion 324. In these and other embodiments, during delivery of the IOL 320, the first opposing portion 322 of the optic 321 may unfold faster than the second opposing portion 324 of the optic 321 because the first opposing portion 322 is coupled to the leading haptic 328, which enters the surgical site first. The second opposing portion 324 is coupled to the trailing haptic 326, which enters the surgical site last. The second opposing portion 324 may unfold slower than the first opposing portion 322 because the trailing haptic 326 is contained within the IOL injector 300 for a short period while the leading haptic 328 has already exited the IOL injector 300 through the opening 305. Thus, if the optic fold 330 is positioned vertically (e.g., at the 12 o’clock position) so as to be aligned with the vertical plane 332, the IOL 320 may be tilted upon delivery in the direction of the first opposing portion 322. In these embodiments, the folding chamber 312 may be shaped to offset the optic fold 330 toward the second sidewall 336 to compensate for the faster unfolding of the first opposing portion 322, which may mitigate tilting of the IOL 320 upon delivery.

In some embodiments, the folding chamber 312 may be shaped to offset the optic fold 330 about 15 degrees, about 20 degrees, about 30 degrees, about 40 degrees, about 45 degrees, about 50 degrees, about 60 degrees, about 75 degrees, or other values from the vertical plane 332 toward the second sidewall 336. In some embodiments, the folding chamber 312 may be shaped to offset the optic fold 330 between about 15 degrees and about 75 degrees from the vertical plane 332 toward the second sidewall 336. In some embodiments, the folding chamber 312 may be shaped to offset the optic fold 330 between about 30 degrees and about 60 degrees from the vertical plane 332 toward the second sidewall 336. In some embodiments, the folding chamber 312 may be shaped to offset the optic fold 330 less than 15 degrees from the vertical plane 332 toward the second sidewall 336. In some embodiments, the folding chamber 312 may be shaped to offset the optic fold 330 from the vertical plane 332 toward the second sidewall 336 in any range using any of the preceding values as endpoints, including ranges that include the value as an endpoint and the vertical plane 332 as an endpoint (e.g., less than 15 degrees, less than 30 degrees, less than 60 degrees, between 15 degrees and 45 degrees, between 20 degrees and 50 degrees, etc.).

The components of IOL injector 300 described above may be similar to and perform similar functions as similarly named components described throughout the description and vice versa. For example, the injector body 302, the nozzle 304, and the compartment 306 may be similar to and perform similar functions as the injector body 110, the nozzle 112, and the compartment 116 described with reference to FIGS. 1A–1B. Furthermore, the IOL 320 may be similar to and perform similar functions as the IOL 200 described with reference to FIG. 2. Additionally, the folding chamber 312 may include any or all of the features described with reference to FIGS. 4A–4C.

Modifications, additions, or omissions may be made to the IOL injector 300 of FIGS. 3A-3D without departing from the scope of the present disclosure. For example, in some embodiments, the IOL injector 300 may further include a handpiece body such as the handpiece body 130 illustrated in FIG. 1B. In some of these embodiments, the handpiece body may be coupled to the injector body 302. In some of these embodiments, the moveable plunger 340 may be disposed at least partially within the handpiece body and may be axially translatable relative to the handpiece body such that the moveable plunger 340 advances the IOL 320 from the compartment 306 through the folding chamber 312. In some embodiments, the injector body 302 may be integral with the handpiece body. In some embodiments, the handpiece body may be detachable from the injector body 302. In these embodiments, the injector body 302 may be coupled to a handpiece body via a male-female connection like a pin-and-box, tab-and-recess, tongue-and-groove, key-and-keyseat, or the like. As another example, the first set of tabs 308 and/or the second set of tabs 310 may be omitted.

FIG. 4A–4D illustrate various cross-sectional views of an example intraocular lens (IOL) injector 400. FIG. 4A illustrates a top, cross-sectional view of the IOL injector 400. The IOL injector 400 includes an injector body 402 and a nozzle 430 coupled to the injector body 402. The injector body 402 includes a compartment 406 to house an IOL and receive a moveable plunger. The injector body 402 also includes a folding chamber 404 coupled to the compartment 406. The folding chamber 404 is shaped to fold opposing portions of the IOL as the moveable plunger advances the IOL through the folding chamber 404 to form an optic fold and to offset the optic fold from a vertical plane 420 centered within and extending through the folding chamber 404 and/or the nozzle 430. In some embodiments, the folding chamber 404 may include a first sidewall 412, a second sidewall 414, a third sidewall (not shown in FIGS. 4A–4B), and a fourth sidewall (not shown in FIGS. 4A–4B).

As shown in FIG. 4A, the IOL injector 400 may be a cartridge that may include a first set of tabs 408 and a second set of tabs 410. In some of these embodiments, each tab of the first set of tabs 408 may extend from opposing sides of the injector body 402. In some embodiments, each tab of the second set of tabs 410 may extend from opposing sides of the injector body 402. As described previously, the first set of tabs 408 and the second set of tabs 410 may couple the IOL injector 400 to a handpiece body.

In operation, the IOL such as the IOL 200 of FIG. 2 may be advanced by the moveable plunger from the compartment 406, through the folding chamber 404, into the nozzle 430, and delivered through an opening 405 of the nozzle 430 into, for example, a human eye.

The folding chamber 404 may be shaped to offset the optic fold of the IOL from the vertical plane 420 toward the first sidewall 412 or the second sidewall 414 as the moveable plunger advances the IOL through the folding chamber 404.In some embodiments, the folding chamber 404 may be shaped to offset the optic fold about 15 degrees, about 20 degrees, about 30 degrees, about 40 degrees, about 45 degrees, about 50 degrees, about 60 degrees, about 75 degrees, or other values from the vertical plane 420 toward the first sidewall 412 or the second sidewall 414. In some embodiments, the folding chamber 404 may be shaped to offset the optic fold between about 15 degrees and about 75 degrees from the vertical plane 420 toward the first sidewall 412 or the second sidewall 414. In some embodiments, the folding chamber 404 may be shaped to offset the optic fold between about 30 degrees and about 60 degrees from the vertical plane 420 toward the first sidewall 412 or the second sidewall 414. In some embodiments, the folding chamber 404 may be shaped to offset the optic fold less than 15 degrees from the vertical plane 420 toward the first sidewall 412 or the second sidewall 414. Additionally or alternatively, the folding chamber 404 may be shaped to offset the optic fold from the vertical plane 420 toward the first sidewall 412 or the second sidewall 414 in any range using any of the preceding values as endpoints, including ranges that include the value as an endpoint and the vertical plane 420 as an endpoint (e.g., less than 15 degrees, less than 30 degrees, less than 60 degrees, between 15 degrees and 45 degrees, between 20 degrees and 50 degrees, etc.).

In some embodiments, the folding chamber 404 may be shaped to offset the optic fold from the vertical plane 420 toward the first sidewall 412 or the second sidewall 414 by utilizing different slopes for the first sidewall 412 and the second sidewall 414. For example, the first sidewall 412 may have a slope configuration such that a portion of the IOL contacting the first sidewall 412 folds at a greater rate than an opposing portion of the IOL contacting the second sidewall 414 causing the optic fold to be offset from the vertical plane 420 toward the second sidewall 414. In another example, the second sidewall 414 may have a slope configuration such that a portion of the IOL contacting the second sidewall 414 folds at a greater rate than an opposing portion of the IOL contacting the first sidewall 412 causing the optic fold to be offset from the vertical plane 420 toward the first sidewall 412. In these and other embodiments, the first sidewall 412 and/or the second sidewall 414 may have multiple slopes.

In some embodiments, and as shown in FIG. 4A, at a proximal end of the folding chamber 404, the first sidewall 412 may have a slope of around zero or a negligible slope (e.g., when progressing from the proximal end of the folding chamber 404 towards the distal end of the folding chamber 404, the first sidewall 412 remains at a relatively constant distance from the vertical plane 420). In contrast, the second sidewall 414 may have a greater slope at the proximal end of the folding chamber 404 than the first sidewall 412 (e.g., when progressing from the proximal end of the folding chamber 404 towards the distal end of the folding chamber 404, the second sidewall 414 progressively draws closer to the vertical plane 420). As a result, a distance 422 between the first sidewall 412 and the vertical plane 420 may be greater than a distance 424 between the second sidewall 414 and the vertical plane 420 from the proximal end of the folding chamber 404 to the distal end of the folding chamber 404 (represented by the length (L) in FIG. 4A). In some embodiments, the second sidewall 414 may have a greater slope than the first sidewall 412 at the proximal end of the folding chamber 404, but the first sidewall 412 may have a greater slope at the distal end of the folding chamber 404. In some of these embodiments, the distance 422 between the first sidewall 412 and the vertical plane 420 may be greater than the distance 424 between the second sidewall 414 and the vertical plane 420 for a portion of the length (L) of the folding chamber 404.

In these and other embodiments, there is less space in the folding chamber 404 between the vertical plane 420 and the second sidewall 414 than there is between the vertical plane 420 and the first sidewall 412 for at least a portion of the folding chamber 404. In operation, for the length of the folding chamber 404 where the distance 422 is greater than the distance 424, the second sidewall 414 causes the portion of the IOL contacting the second sidewall 414 to fold at a greater rate than the portion of the IOL contacting the first sidewall 412 such that the optic fold created by opposing portions of the IOL is offset from the vertical plane 420 toward the first sidewall 412 (e.g., in the direction indicated by arrow 421).

In some embodiments, the first sidewall 412 may have a greater slope at the proximal end of the folding chamber 404. As a result, the distance 422 between the first sidewall 412 and the vertical plane 420 may be less than the distance 424 between the second sidewall 414 and the vertical plane 420 from the proximal end of the folding chamber 404 to the distal end of the folding chamber 404. In some embodiments, the first sidewall 412 may have a greater slope than the second sidewall 414 at the proximal end of the folding chamber 404, but the second sidewall 414 may have a greater slope at the distal end of the folding chamber 404. In some of these embodiments, the distance 422 between the first sidewall 412 and the vertical plane 420 may be less than the distance 424 between the second sidewall 414 and the vertical plane 420 for a portion of the length (L) of the folding chamber 404.

In these and other embodiments, there is less space in the folding chamber 404 between the vertical plane 420 and the first sidewall 412 than there is between the vertical plane 420 and the second sidewall 414 for at least a portion of the folding chamber 404. In operation, for the length of the folding chamber 404 where the distance 422 is less than the distance 424, the first sidewall 412 causes the portion of the IOL contacting the first sidewall 412 to fold at a greater rate than the portion of the IOL contacting the second sidewall 414 such that the optic fold created by opposing portions of the IOL is offset from the vertical plane toward the second sidewall 414 (e.g. in the direction opposite of that shown by arrow 421).

In some embodiments, the first sidewall 412 may extend inwards toward the vertical plane 420 at a lesser slope than the second sidewall 414. For example, the first sidewall 412 may narrow toward the vertical plane 420 at a lesser rate than the second sidewall 414 narrows toward the vertical plane 420 over the length (L) of the folding chamber 404. In some embodiments, the first sidewall 412 may extend inwards toward the vertical plane 420 at a lesser slope than the second sidewall 414 for a portion of the length (L) of the folding chamber 404. In some embodiments, due to the first sidewall 412 having a lesser slope than the second sidewall 414, a portion of the IOL may be folded inwards by the second sidewall 414 at a greater rate than an opposing portion of the IOL is folded inwards by the first sidewall 412. As a result, when the optic fold is formed, the optic fold may be offset from the vertical plane 420 toward the first sidewall 412 (e.g., in the direction represented by arrow 421) due to the shape of the folding chamber 404.

As shown in FIG. 4A, although the first sidewall 412 may have a lesser slope than the second sidewall 414 at the proximal end of the folding chamber 404, the first sidewall 412 and the second sidewall 414 may ultimately extend inwards toward the vertical plane 420 and narrow the folding chamber 404 by the same amount over the length (L) of the folding chamber 404. For example, the second sidewall 414 may have a greater slope than the first sidewall 412 at the proximal end of the folding chamber 404, but the first sidewall 412 may have a greater slope than the second sidewall 414 at a more distal portion of the folding chamber 404 such that the distance 422 and the distance 424 are equivalent or nearly equivalent at the distal end of the folding chamber 404. However, because the second sidewall 414 may have a greater slope at the proximal end of the folding chamber 404, the distance 424 between the second sidewall 414 and the vertical plane 420 may remain less than the distance 422 between the first sidewall 412 and the vertical plane 420 for the length (L) of the folding chamber 404 until the distal end of the folding chamber 404 where the distance 422 and the distance 424 may be equivalent or nearly equivalent.

Thus, the second sidewall 414 may fold a portion of the IOL inwards toward the vertical plane 420 at a greater rate than the first sidewall 412 may fold an opposing portion of the IOL toward the vertical plane 420. As a result, when the optic fold is formed, the optic fold may be offset from the vertical plane 420 toward the first sidewall 412.

In some embodiments, the first sidewall 412 may be sloped for only a portion of the length (L) of the folding chamber 404. For example, as shown in FIG. 4A, the first sidewall 412 may have a slope of around zero at a proximal portion of the folding chamber 404, and then may extend inwards toward the vertical plane 420 at a more distal portion of the folding chamber 404. In some embodiments, the second sidewall 414 may be sloped for only a portion of the length (L) of the folding chamber 404. For example, the second sidewall 414 may have a nonzero slope such that the second sidewall 414 extends inwards toward the vertical plane 420 at a proximal portion of the folding chamber 404, and then may taper off such that the second sidewall 414 has a lesser slope at a more distal portion of the folding chamber 404.

In some embodiments, inverse to the folding chamber 404 illustrated in FIGS. 4A-4D, the slopes of the first sidewall 412 and the second sidewall 414 may be reversed to accommodate different IOL configurations and to offset the optic fold towards the second sidewall 414 rather than the first sidewall 412. For example, in some embodiments, the second sidewall 414 may extend inwards toward the vertical plane 420 at a lesser slope than the first sidewall 412. For example, the second sidewall 414 may narrow toward the vertical plane 420 at a lesser rate than the first sidewall 412 narrows toward the vertical plane 420 over the length (L) of the folding chamber 404. In some embodiments, the second sidewall 414 may extend inwards toward the vertical plane 420 at a lesser slope than the first sidewall 412 for a portion of the length (L) of the folding chamber 404. In some embodiments, a portion of the IOL may be folded inwards by the first sidewall 412 at a greater rate than an opposing portion of the IOL is folded inwards by the second sidewall 414 because the first sidewall 412 extends inwards at a greater slope than the second sidewall 414. As a result, when the optic fold is formed, the optic fold may be offset from the vertical plane 420 toward the second sidewall 414 (e.g., in the direction opposite of that represented by arrow 421) due to the shape of the folding chamber 404.

Although the second sidewall 414 may have a lesser slope than the first sidewall 412 at the proximal end of the folding chamber 404, the first sidewall 412 and the second sidewall 414 may ultimately extend inwards toward the vertical plane 420 and narrow the folding chamber 404 by the same amount over the length (L) of the folding chamber 404. For example, the first sidewall 412 may have a greater slope than the second sidewall 414 at the proximal end of the folding chamber 404, but the second sidewall 414 may have a greater slope than the first sidewall 412 at a more distal portion of the folding chamber 404 such that the distance 422 and the distance 424 are equivalent or nearly equivalent at the distal end of the folding chamber 404. However, because the first sidewall 412 may have a greater slope at the proximal end of the folding chamber 404, the distance 422 between the first sidewall 412 and the vertical plane 420 may remain less than the distance 424 between the second sidewall 414 and the vertical plane 420 for the length (L) of the folding chamber 404 until the distal end of the folding chamber 404 where the distance 422 and the distance 424 may be equivalent or nearly equivalent.

Thus, the first sidewall 412 may fold a portion of the IOL inwards toward the vertical plane 420 at a greater rate the second sidewall 414 may fold an opposing portion of the IOL inwards toward the vertical plane 420. As a result, when the optic fold is formed, the optic fold may be offset from the vertical plane 420 toward the second sidewall 414.

FIG. 4B illustrates a top, cross-sectional view of the IOL injector 400 and demonstrates a modification to the first sidewall 412 compared to FIG. 4A. As shown in FIGS. 4A and 4B, the second sidewall 414 may have a greater slope than the first sidewall 412 at a proximal end of the folding chamber 404. As The greater slope of the second sidewall 414 reduces the distance 424 between the second sidewall 414 and the vertical plane 420. At the proximal end of the folding chamber 404, the first sidewall 412 may have a zero slope (similar to that of FIG. 4A). However, unlike FIG. 4A, the first sidewall 412 may have a first slope 432 for a portion of the folding chamber 404 and a second slope 434 for a portion of the folding chamber 404. The first slope 432 may be at a more proximal portion of the folding chamber 404 than the second slope 434. The first slope 432 may be less compared to the slope of the first sidewall 412 shown in FIG. 4A, and the second slope 434 may be greater than the slope of the first sidewall 412 shown in FIG. 4A. In these embodiments, the first sidewall 412 having a lesser slope (compared to FIG. 4A) where the first sidewall 412 begins to extend inwards toward the vertical plane 420 increases the distance 422 between the first sidewall 412 and the vertical plane 420 relative to FIG. 4A. As a result, the IOL may have an increased response to the slope of the second sidewall 414 such that the second sidewall 414 may fold a portion of the IOL inwards toward the vertical plane 420 more so than in FIG. 4A. Thus, when the optic fold is formed, the optic fold may be offset from the vertical plane 420 toward the first sidewall 412 (e.g., in the direction of arrow 421) more in the embodiment illustrated in FIG. 4B than in the embodiment illustrated in FIG. 4A.

In embodiments where an offset toward the first sidewall 412 is desired, the second sidewall 414 may be modified in a similar fashion and the first sidewall 412 may have a greater slope than the second sidewall 414 at the proximal end of the folding chamber 404 (e.g. a mirror image of the IOL injector 400 shown in FIG. 4B). Modifying the second sidewall 414 to include the first slope 432 and the second slope 434 increases the distance 424 between the second sidewall 414 and the vertical plane 420 and, in these embodiments, the IOL may have an increased response to the slope of the first sidewall 412. Thus, when the optic fold is formed, the optic fold may be offset from the vertical plane 420 toward the second sidewall 414 (e.g., in the direction opposite of that shown by arrow 421).

FIG. 4C illustrates a cross-sectional view of the IOL injector 400 taken along the 4C–4C cutting plane of FIG. 4A. In some embodiments, at least a portion of one or both of the first sidewall 412 or the third sidewall 416 may include or be shaped to define a protrusion 426 extending into the folding chamber 404 on the same side of the vertical plane 420 as the first sidewall 412. In some embodiments, at least a portion of the first sidewall 412 and/or the third sidewall 416 may include or be shaped to define the protrusion 426 for a portion of the length (L) of the folding chamber 404. In these and other embodiments, the protrusion 426 may limit and/or delay the folding progression of a portion of the IOL contacting the first sidewall 412. For example, the protrusion 426 may delay the folding of the portion of the IOL contacting the first sidewall 412 in comparison to an opposing portion of the IOL which may be folded inwards toward the vertical plane 420 by the second sidewall 414. Furthermore, in some embodiments, the second sidewall 414 may curve inward at a greater rate than the first sidewall 412 (e.g., as shown in FIG. 4D). Thus, even though the opposing portions may be folded at different times, the folding progression of the portion of the IOL contacting the first sidewall 412 may be less than the folding progression of the portion of the IOL contacting the second sidewall 414. As a result, when the optic fold is formed, the optic fold may be offset from the vertical plane 420 toward the first sidewall 412 (e.g., in the direction of arrow 421).

In some embodiments, inverse to the folding chamber 404 illustrated in FIG. 4C, the protrusion 426 may be located on an opposite side of the folding chamber 404 to accommodate different IOL configurations and to offset the optic fold towards the second sidewall 414 rather than the first sidewall 412. For example, at least a portion of one or both of the second sidewall 414 or the third sidewall 416 may include or be shaped to define the protrusion 426 extending into the folding chamber 404 on the same side of the vertical plane 420 as the second sidewall 414. In some embodiments, at least a portion of the second sidewall 414 and/or the third sidewall 416 may include or be shaped to define the protrusion 426 for a portion of the length (L) of the folding chamber 404. In these and other embodiments, the protrusion 426 may limit and/or delay the folding progression of a portion of the IOL contacting the second sidewall 414. For example, the protrusion 426 may delay the folding of the portion of the IOL contacting the second sidewall 414 in comparison to an opposing portion of the IOL which may be folded inwards toward the vertical plane 420 by the first sidewall 412. Furthermore, in some embodiments, the first sidewall 412 may curve inward at a greater rate than the second sidewall 414 (e.g., inverse of FIG. 4D). Thus, even though the opposing portions may be folded at different times, the folding progression of the portion of the IOL contacting the second sidewall 414 may be less than the folding progression of the portion of the IOL contacting the first sidewall 412. As a result, when the optic fold is formed, the optic fold may be offset from the vertical plane 420 toward the second sidewall 414 (e.g., in the direction opposite of arrow 421).

FIG. 4D illustrates a cross-sectional view of the IOL injector 400 taken along the 4D–4D cutting plane of FIG. 4A. As shown in FIG. 4D, in some embodiments, a first portion 428 defined by one or both of the second sidewall 414 or the third sidewall 416 may curve inward toward the vertical plane 420 at a greater rate than a second portion 429 defined by one or both of the first sidewall 412 or the third sidewall 416. For example, the first portion 428 may have a tighter radius of curvature than the second portion 429 such that the first portion 428 may curve inward toward the vertical plane 420 at a greater rate than the second portion 429. In some embodiments, the first portion 428 may curve inward toward the vertical plane 420 at a greater rate than the second portion 429 for a portion of the length (L) of the folding chamber 404. The first portion 428 curving inwardly at a greater rate than the second portion 429 may cause a portion of the IOL to be folded inwards toward the vertical plane 420 at a greater rate than the second portion 429 causes an opposing portion of the IOL to be folded inwards toward the vertical plane 420. Thus, when the optic fold is formed, the optic fold may be offset from the vertical plane 420 toward the first sidewall 412 (e.g., in the direction of arrow 421).

In some embodiments, the IOL injector 400 may be configured inverse of that illustrated in FIG. 4D to accommodate different IOL configurations and to offset the optic fold towards the second sidewall 414 rather than the first sidewall 412. For example, the second portion 429 may curve inward toward the vertical plane 420 at a greater rate than the first portion 428. For instance, the second portion 429 may have a tighter radius of curvature than the first portion 428 such that the second portion 429 may curve inward toward the vertical plane 420 at a greater rate than the first portion 428. As a result, the second portion 429 may cause a portion of the IOL to be folded inwards toward the vertical plane 420 at a greater rate than the first portion 428 causes an opposing portion of the IOL to be folded inwards toward the vertical plane 420. As a result, when the optic fold is formed, the optic fold may be offset from the vertical plane 420 toward the second sidewall 414 (e.g., in the direction opposite of that of arrow 421).

In some embodiments, a portion of the third sidewall 416 on the same side of the vertical plane 420 as the first sidewall 412 may at least partially define the second portion 429. In some embodiments, a portion of the third sidewall 416 on the same side of the vertical plane 420 as the second sidewall 414 may at least partially define the first portion 428.

In some embodiments, the folding chamber 404 may have a substantially rounded cross-section such that each of the interfaces between the first sidewall 412 the second sidewall 414, the third sidewall 416 and the fourth sidewall 418 are rounded.

In some embodiments, even if the first sidewall 412 and the second sidewall 414 permit the two sides of the IOL to fold at the same time (not shown), by utilizing a different radius of curvature on one side, the side with the tighter radius of curvature may result in that side of the optic folding more quickly such that the optic fold is offset from the vertical plane 420.

Modifications, additions, or omissions may be made to the IOL injector 400 of FIGS. 4A–4D without departing from the scope of the present disclosure. For example, in some embodiments, the first set of tabs 408 and/or the second set of tabs 410 may be omitted. In some embodiments, the IOL injector 400 may further include a handpiece body coupled to the injector body 402. In some of these embodiments, the handpiece body may be detachable from the injector body 402. In some embodiments, the IOL injector 400 may further include the moveable plunger disposed at least partially with the handpiece body. In these embodiments, the moveable plunger may be axially translatable relative to the handpiece body such that the moveable plunger advances the IOL from the compartment 406 through the folding chamber 404. Furthermore, the IOL injector 400 may be shaped in any fashion which may offset the optic fold from the vertical plane 420. Thus, the IOL injector 400 may include any combination of the features described in FIGS. 4A-D or omit these features entirely if the folding chamber 404 is otherwise shaped to offset the optic fold from the vertical plane.

FIG. 5 is a flow diagram illustrating a method 500 for delivering an IOL to a surgical site, in accordance with one or more embodiments of the present disclosure. One or more operations of the method 500 may be performed by any suitable system, apparatus, or device, such as the IOL injector 100, 300, or 400 respectively described with reference to FIGS. 1A–1B, FIGS. 3A–3D, and FIGS. 4A–4D.

At block 510, a nozzle of an intraocular lens injector may be positioned proximate a surgical site. For example, any one of nozzle 112 of IOL injector 100 illustrated in FIGS. 1A-B, nozzle 304 of IOL injector 300 illustrated in FIGS. 3A–3D or nozzle 430 of IOL injector 400 illustrated in FIGS. 4A–4D may be placed proximate a surgical site.

At block 520, an IOL may be folded such that opposing portions of the IOL form an optic fold that is offset from a vertical plane centered within and extending through the nozzle. The intraocular lens injector may include similar components as, and function similarly to, the IOL injectors 100, 300, or 400 respectively described with reference to FIGS. 1A–1B, FIGS. 3A–3D, and FIGS. 4A–4D. For example, as illustrated by FIGS. 3A-3D, an IOL the same as or similar to the IOL 320 may be advanced by a moveable plunger the same as or similar to the moveable plunger 340 through a folding chamber the same as or similar to the folding chamber 312 of IOL injector 300 such that opposing portions of the IOL form an optic fold that is offset from a vertical plane centered within and extending through the folding chamber and/or the nozzle, which may be the same as or similar to the nozzle 304 of FIG. 3.

In some embodiments, the IOL injector may include a folding chamber that may be defined in part by a first sidewall, a second sidewall opposite the first sidewall, a third sidewall extending between the first sidewall and the second sidewall, and a fourth sidewall opposite the third sidewall and extending between the first sidewall and the second sidewall. The sidewalls may be similar to the sidewalls described previously with reference to FIGS. 3A–3D and/or FIGS. 4A–4D. In some embodiments, the folding of the IOL may be performed using edge rollers and/or haptic rollers. In some embodiments, the method 500 may further include activating a plunger to advance the IOL into the folding chamber, and the folding chamber may be shaped to fold the opposing portions of the IOL as the plunger advances the IOL through the folding chamber such that the optic fold is formed and offset from the vertical plane.

In some embodiments, the folding chamber may be shaped to offset the optic fold about 15 degrees, about 20 degrees, about 30 degrees, about 40 degrees, about 45 degrees, about 50 degrees, about 60 degrees, about 75 degrees, or other values from the vertical plane toward the first sidewall or the second sidewall. In some embodiments, the folding chamber may be shaped to offset the optic fold between about 15 degrees and about 75 degrees from the vertical plane toward the first sidewall or the second sidewall. In some embodiments, the folding chamber may be shaped to offset the optic fold between about 30 degrees and about 60 degrees from the vertical plane toward the first sidewall or the second sidewall. In some embodiments, the folding chamber may be shaped to offset the optic fold less than 15 degrees from the vertical plane toward the first sidewall or the second sidewall. Additionally or alternatively, the folding chamber may be shaped to offset the optic fold from the vertical plane toward the first sidewall or the second sidewall in any range using any of the preceding values as endpoints, including ranges that include the value as an endpoint and the vertical plane as an endpoint (e.g., less than 15 degrees, less than 30 degrees, less than 60 degrees, between 15 degrees and 45 degrees, between 20 degrees and 50 degrees, etc.).

In some embodiments, the second sidewall may have a greater slope than the first sidewall for at least a portion of the folding chamber such that a first distance between the vertical plane and the first sidewall may be greater than a second distance between the vertical plane and the second sidewall. In these and other embodiments, the second sidewall may be configured to offset the optic fold from the vertical plane toward the first sidewall. For example, as illustrated in FIG. 4A, the second sidewall 414 may have a greater slope than the first sidewall 412 for at least a portion of the folding chamber 404 such that the distance 422 between the first sidewall 412 and the vertical plane 420 may be greater than the distance 424 in the folding chamber 404 between the second sidewall 414 and the vertical plane 420.

In some embodiments, inverse to the folding chamber illustrated in FIG. 4A, the first sidewall may have a greater slope than the second sidewall for at least a portion of the folding chamber such that a first distance between the vertical plane and the first sidewall may be less than a second distance between the vertical plane and the second sidewall. In these and other embodiments, the first sidewall may be configured to offset the optic fold from the vertical plane toward the second sidewall. For example, the first sidewall 412 may have a greater slope than the second sidewall 414 for at least a portion of the folding chamber 404 such that the distance 422 between the first sidewall 412 and the vertical plane 420 may be less than the distance 424 in the folding chamber 404 between the second sidewall 414 and the vertical plane 420.

In some embodiments, the folding chamber may include a first portion defined by one or both of the second sidewall or the third sidewall and a second portion defined by one or both of the first sidewall or the third sidewall. In these and other embodiments, the first portion may curve inward toward the vertical plane at a greater rate than the second portion such that the first portion is configured to offset the optic fold from the vertical plane toward the first sidewall. For example, the first portion 428 may curve inward toward the vertical plane 420 at a greater rate than the second portion 429 as described with reference to FIG. 4D.

In some embodiments, inverse to the folding chamber 404 illustrated in FIG. 4D, the folding chamber may include a first portion defined by one or both of the second sidewall or the third sidewall and a second portion defined by one or both of the first sidewall or the third sidewall. In these and other embodiments, the second portion may curve inward toward the vertical plane at a greater rate than the first portion such that the second portion is configured to offset the optic fold from the vertical plane toward the second sidewall. For example, the second portion 429 may curve inward toward the vertical plane 420 at a greater rate than the first portion 428.

In some embodiments, the first sidewall may extend inwards toward the vertical plane at a lesser slope than the second sidewall. In these and other embodiments, the first sidewall may be configured to offset the optic fold from the vertical plane toward the first sidewall. For example, as illustrated in FIG. 4A, the first sidewall 412 may extend inward toward the vertical plane 420 at a lesser slope than the second sidewall 414.

In some embodiments, inverse to the folding chamber illustrated in FIG. 4A, the second sidewall may extend inwards toward the vertical plane at a lesser slope than the first sidewall. In these and other embodiments, the second sidewall may be configured to offset the optic fold from the vertical plane toward the second sidewall. For example, the second sidewall 414 may extend inward toward the vertical plane 420 at a lesser slope than the first sidewall 412.

In some embodiments, a protrusion may extend from at least a portion of one or both of the first sidewall or the third sidewall on the same side of the vertical plane as the first sidewall. In these and other embodiments, the protrusion may be configured to offset the optic fold from the vertical plane toward the first sidewall. The protrusion may be similar to the protrusion 426 described with reference to FIG. 4C.

In some embodiments, inverse to the folding chamber illustrated in FIG. 4C, a protrusion may extend from at least a portion of one or both of the second sidewall or the third sidewall on the same side of the vertical plane as the second sidewall. In these and other embodiments, the protrusion may be configured to offset the optic fold from the vertical plane toward the second sidewall. The protrusion may be similar to the protrusion 426 described with reference to FIG. 4C but on the opposite of the injector body.

At block 530, the IOL may be delivered to the surgical site. For example, the IOL 200 of FIG. 2 or the IOL 320 of FIG. 3 may be delivered to the surgical site by the IOL injector 100 of FIG. 1, the IOL injector 300 of FIGS. 3A–3D, or the IOL injector 400 of FIGS. 4A–4D. Delivering the IOL into the surgical site may be performed by advancing a plunger to eject the IOL out of the nozzle. The plunger may be advanced after the IOL has been folded to deliver the IOL into the surgical site.

Modifications, additions, or omissions may be made to the method of FIG. 5 without departing from the scope of the present disclosure. For example, in some embodiments, the IOL injector may further include a handpiece body coupled to the injector body. In some of these embodiments, the handpiece body may be detachable from the injector body. In some embodiments, the IOL injector may further include a moveable plunger disposed at least partially with the handpiece body. The moveable plunger may be axially translatable relative to the handpiece body such that the moveable plunger advances the IOL through the folding chamber.

While examples are provided herein of a folding chamber where sidewalls push against an optic of an IOL to fold the optic such that the optic fold is offset from a vertical plane, it will be appreciated that other folding methods known in the art are contemplated that may result in the optic fold being offset from the vertical plane. For example, haptic folders and edge rollers may be used to fold the haptics and/or optics and may be positioned, shaped, or sized such that when the folding is completed the optical fold is offset from the vertical plane. An example of such folding is described in US Pat. No. 11,801,130, the entire disclosure of which is hereby incorporated by reference for all that it teaches. As used herein, a recitation of “and/or” with respect to two or more elements should be interpreted to mean only one element, or a combination of elements. For example, “element A, element B, and/or element C” may include only element A, only element B, only element C, element A and element B, element A and element C, element B and element C, or elements A, B, and C. In addition, “at least one of element A or element B” may include at least one of element A, at least one of element B, or at least one of element A and at least one of element B. Further, “at least one of element A and element B” may include at least one of element A, at least one of element B, or at least one of element A and at least one of element B. Additionally, use of the term “based on” should not be interpreted as “only based on” or “based only on.” Rather, a first element being “based on” a second element includes instances in which the first element is based on the second element but may also be based on one or more additional elements.

The subject matter of the present disclosure is described with specificity herein to meet statutory requirements. However, the description itself is not intended to limit the scope of this disclosure. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

The subject technology of the present disclosure is illustrated, for example, according to various aspects described below. Various examples of aspects of the present disclosure are described as numbered examples (1, 2, 3, etc.) for convenience. These are provided as examples and do not limit the present disclosure. The aspects of the various implementations described herein may be omitted, substituted for aspects of other implementations, or combined with aspects of other implementations unless context dictates otherwise. For example, one or more aspects of example 1 below may be omitted, substituted for one or more aspects of another example (e.g., example 2) or examples, or combined with aspects of another example. The following is a non-limiting summary of some example implementations presented herein.

Example 1. An intraocular lens injector comprising:

an injector body comprising:

a compartment to house an intraocular lens (IOL) and receive a moveable plunger;

a folding chamber coupled to the compartment, the folding chamber shaped to fold opposing portions of the IOL as the moveable plunger advances the IOL through the folding chamber to form an optic fold and to offset the optic fold from a vertical plane centered within and extending through the folding chamber; and

a nozzle coupled to the injector body and configured to deliver the IOL to a surgical site.

Example 2. The intraocular lens injector of Example 1, wherein the folding chamber is defined, at least in part, by:

a first sidewall;

a second sidewall opposite the first sidewall;

a third sidewall extending between the first sidewall and the second sidewall; and

a fourth sidewall opposite the third sidewall and extending between the first sidewall and the second sidewall.

Example 3. The intraocular lens injector of Example 1 or Example 2, wherein the folding chamber is shaped to offset the optic fold between about 15 degrees and about 75 degrees from the vertical plane toward the first sidewall.

Example 4. The intraocular lens injector of Example 1 or Example 2, wherein the folding chamber is shaped to offset the optic fold between about 30 degrees and about 60 degrees from the vertical plane toward the first sidewall.

Example 5. The intraocular lens injector of any of Examples 2–4, wherein the second sidewall has a greater slope than the first sidewall for at least a portion of the folding chamber such that a first distance between the vertical plane and the first sidewall is greater than a second distance between the vertical plane and the second sidewall, the second sidewall configured to offset the optic fold from the vertical plane toward the first sidewall.

Example 6. The intraocular lens injector of any of Examples 2–5, wherein the folding chamber includes:

a first portion defined by one or both of the second sidewall or the third sidewall; and

a second portion defined by one or both of the first sidewall or the third sidewall, the first portion curving inward toward the vertical plane at a greater rate than the second portion such that the first portion is configured to offset the optic fold from the vertical plane toward the first sidewall.

Example 7. The intraocular lens injector of any of Examples 2–6, wherein the first sidewall extends inwards toward the vertical plane at a lesser slope than the second sidewall, the first sidewall configured to offset the optic fold from the vertical plane toward the first sidewall.

Example 8. The intraocular lens injector of any of Examples 2–7, wherein a protrusion extends from at least a portion of one or both of the first sidewall or the third sidewall on the same side of the vertical plane as the first sidewall, the protrusion configured to offset the optic fold from the vertical plane toward the first sidewall.

Example 9. The intraocular lens injector of Example 1 or Example 2, wherein the folding chamber is shaped to offset the optic fold between about 15 degrees and about 75 degrees from the vertical plane toward the second sidewall.

Example 10. The intraocular lens injector of Examples 1–2 or Example 9, wherein the folding chamber is shaped to offset the optic fold between about 30 degrees and about 60 degrees from the vertical plane toward the second sidewall.

Example 11. The intraocular lens injector of any of Example 2 or Examples 9–10, wherein the first sidewall has a greater slope than the second sidewall for at least a portion of the folding chamber such that a first distance between the vertical plane and the first sidewall is less than a second distance between the vertical plane and the second sidewall, the first sidewall configured to offset the optic fold from the vertical plane toward the second sidewall.

Example 12. The intraocular lens injector of any of Example 2 or Examples 9–11, wherein the folding chamber includes:

a first portion defined by one or both of the second sidewall or the third sidewall; and

a second portion defined by one or both of the first sidewall or the third sidewall, the second portion curving inward toward the vertical plane at a greater rate than the first portion such that the second portion is configured to offset the optic fold from the vertical plane toward the second sidewall.

Example 13. The intraocular lens injector of any of Example 2 or Examples 9–12, wherein the second sidewall extends inwards toward the vertical plane at a lesser slope than the first sidewall, the second sidewall configured to offset the optic fold from the vertical plane toward the second sidewall.

Example 14. The intraocular lens injector of any of Example 2 or Examples 9–13, wherein a protrusion extends from at least a portion of one or both of the second sidewall or the third sidewall on the same side of the vertical plane as the second sidewall, the protrusion configured to offset the optic fold from the vertical plane toward the second sidewall.

Example 15. The intraocular lens injector of any of Examples 1–14, further comprising a handpiece body coupled to the injector body

Example 16. The intraocular lens injector Example 15, wherein the handpiece body is detachable from the injector body.

Example 17. The intraocular lens injector of Example 15, further comprising the moveable plunger disposed at least partially within the handpiece body and axially translatable relative to the handpiece body such that the moveable plunger advances the IOL from the compartment through the folding chamber.

Example 18. A method comprising:

positioning a nozzle of an intraocular lens injector proximate a surgical site;

folding an intraocular lens (IOL) such that opposing portions of the IOL form an optic fold that is offset from a vertical plane centered within and extending through the nozzle; and

delivering the IOL to the surgical site.

Example 19. The method of Example 18, further comprising activating a plunger to advance the IOL into a folding chamber, the folding chamber shaped to fold the opposing portions of the IOL as the plunger advances the IOL through the folding chamber such that the optic fold is formed and offset from the vertical plane.

Example 20. The method of Example 18, wherein the folding of the IOL is performed using edge rollers.

Example 21. The method of any of Examples 18–20 performed by any of the intraocular lens injectors of Examples 1–17.

What is claimed is:

1. An intraocular lens injector comprising:
an injector body comprising:
a compartment to house an intraocular lens (IOL) and receive a moveable plunger;
a folding chamber coupled to the compartment, the folding chamber shaped to fold opposing portions of the IOL as the moveable plunger advances the IOL through the folding chamber to form an optic fold and to offset the optic fold from a vertical plane centered within and extending through the folding chamber; and
a nozzle coupled to the injector body and configured to deliver the IOL to a surgical site.

2. The intraocular lens injector of claim 1, wherein the folding chamber is defined, at least in part, by:
a first sidewall;
a second sidewall opposite the first sidewall;
a third sidewall extending between the first sidewall and the second sidewall; and
a fourth sidewall opposite the third sidewall and extending between the first sidewall and the second sidewall.

3. The intraocular lens injector of claim 2, wherein the folding chamber is shaped to offset the optic fold between about 15 degrees and about 75 degrees from the vertical plane toward the first sidewall.

4. The intraocular lens injector of claim 2, wherein the folding chamber is shaped to offset the optic fold between about 30 degrees and about 60 degrees from the vertical plane toward the first sidewall.

5. The intraocular lens injector of claim 2, wherein the second sidewall has a greater slope than the first sidewall for at least a portion of the folding chamber such that a first distance between the vertical plane and the first sidewall is greater than a second distance between the vertical plane and the second sidewall, the second sidewall configured to offset the optic fold from the vertical plane toward the first sidewall.

6. The intraocular lens injector of claim 2, wherein the folding chamber includes:
a first portion defined by one or both of the second sidewall or the third sidewall; and
a second portion defined by one or both of the first sidewall or the third sidewall, the first portion curving inward toward the vertical plane at a greater rate than the second portion such that the first portion is configured to offset the optic fold from the vertical plane toward the first sidewall.

7. The intraocular lens injector of claim 2, wherein the first sidewall extends inwards toward the vertical plane at a lesser slope than the second sidewall, the first sidewall configured to offset the optic fold from the vertical plane toward the first sidewall.

8. The intraocular lens injector of claim 2, wherein a protrusion extends from at least a portion of one or both of the first sidewall or the third sidewall on the same side of the vertical plane as the first sidewall, the protrusion configured to offset the optic fold from the vertical plane toward the first sidewall.

9. The intraocular lens injector of claim 2, wherein the folding chamber is shaped to offset the optic fold between about 15 degrees and about 75 degrees from the vertical plane toward the second sidewall.

10. The intraocular lens injector of claim 2, wherein the folding chamber is shaped to offset the optic fold between about 30 degrees and about 60 degrees from the vertical plane toward the second sidewall.

11. The intraocular lens injector of claim 2, wherein the first sidewall has a greater slope than the second sidewall for at least a portion of the folding chamber such that a first distance between the vertical plane and the first sidewall is less than a second distance between the vertical plane and the second sidewall, the first sidewall configured to offset the optic fold from the vertical plane toward the second sidewall.

12. The intraocular lens injector of claim 2, wherein the folding chamber includes:
a first portion defined by one or both of the second sidewall or the third sidewall; and
a second portion defined by one or both of the first sidewall or the third sidewall, the second portion curving inward toward the vertical plane at a greater rate than the first portion such that the second portion is configured to offset the optic fold from the vertical plane toward the second sidewall.

13. The intraocular lens injector of claim 2, wherein the second sidewall extends inwards toward the vertical plane at a lesser slope than the first sidewall, the second sidewall configured to offset the optic fold from the vertical plane toward the second sidewall.

14. The intraocular lens injector of claim 2, wherein a protrusion extends from at least a portion of one or both of the second sidewall or the third sidewall on the same side of the vertical plane as the second sidewall, the protrusion configured to offset the optic fold from the vertical plane toward the second sidewall.

15. The intraocular lens injector of claim 1, further comprising a handpiece body coupled to the injector body.

16. The intraocular lens injector of claim 15, wherein the handpiece body is detachable from the injector body.

17. The intraocular lens injector of claim 15, further comprising the moveable plunger disposed at least partially within the handpiece body and axially translatable relative to the handpiece body such that the moveable plunger advances the IOL from the compartment through the folding chamber.

18. A method comprising:
positioning a nozzle of an intraocular lens injector proximate a surgical site;
folding an intraocular lens (IOL) such that opposing portions of the IOL form an optic fold that is offset from a vertical plane centered within and extending through the nozzle; and
delivering the IOL to the surgical site.

19. The method of claim 18, further comprising activating a plunger to advance the IOL into a folding chamber, the folding chamber shaped to fold the opposing portions of the IOL as the plunger advances the IOL through the folding chamber such that the optic fold is formed and offset from the vertical plane.

20. An intraocular lens injector comprising:
an injector body comprising:
a compartment to house an intraocular lens (IOL) and receive a moveable plunger;
a folding chamber coupled to the compartment, the folding chamber shaped to fold opposing portions of the IOL as the moveable plunger advances the IOL through the folding chamber to form an optic fold and to offset the optic fold from a vertical plane centered within and extending through the folding chamber, wherein:
the folding chamber is defined, at least in part, by:
a first sidewall;
a second sidewall opposite the first sidewall;
a third sidewall extending between the first sidewall and the second sidewall; and
a fourth sidewall opposite the third sidewall and extending between the first sidewall and the second sidewall;
a handpiece body coupled to the injector body, the moveable plunger being disposed at least partially within the handpiece body and axially translatable relative to the handpiece body such that the moveable plunger advances the IOL from the compartment through the folding chamber; and
a nozzle coupled to the injector body and configured to deliver the IOL to a surgical site.
